(19) United States
(12) Patent Application Publication
(10) Pub. No.: US 2021/0381036 A1
(43) Pub. Date: Dec. 9, 2021

(54) METHODS AND COMPOSITION FOR HIGH THROUGHPUT SINGLE MOLECULE PROTEIN DETECTION SYSTEMS

(71) Applicant: Apton Biosystems, inc., Pleasanton, CA (US)

(72) Inventors: Manohar R. Furtado, San Ramon, CA (US); Bryan P. Staker, San Ramon, CA (US); Niandong Liu, San Ramon, CA (US)

(21) Appl. No.: 16/938,746

(22) Filed: Jul. 24, 2020

Related U.S. Application Data
(63) Continuation of application No. PCT/US2019/015243, filed on Jan. 25, 2019.
(60) Provisional application No. 62/622,053, filed on Jan. 25, 2018.

(51) Int. Cl. C12Q 1/6834; C12Q 1/682; G01N 33/543

(52) U.S. Cl. CPC C12Q 1/6834; G01N 2458/10; G01N 33/54306; C12Q 1/682

(57) ABSTRACT

Disclosed herein are highly multiplexed methods of detecting single target analytes, including complexes, with improved accuracy using a proximity binding assay and single molecule cycled detection.

### CROSS REFERENCE

This application is a continuation of PCT/US2019/015243, filed Jan. 25, 2019, which claims the benefit of U.S. Provisional Application No. 62/622,053, filed Jan. 25, 2018, the contents of which are incorporated herein by reference in their entireties.

### BACKGROUND OF THE INVENTION

Investigative assays for measuring the presence, amount, functional activity, or modifications of a target analyte have become a routine part of modern medical, environmental, pharmaceutical, forensic, and other industrial fields. Examples include commonly used nucleic acid based assays, such as qPCR (quantitative polymerase chain reaction) and DNA microarray, and protein based approaches, such as immunoassay and mass spectrometry. However, various limitations exist in current analyte analysis technologies.

For example, current methods have limited sensitivity and specificity, impacting the accuracy of these methods where analytes are present in biological samples at low copy numbers or in low concentrations. Due to lack of sensitivity, approaches for detection and quantification often require relatively large sample volumes.

Current methods are also limited in their capacity for identification and quantification of a large number of analytes. Simultaneous identification or quantification of multiple different molecular species (e.g., mRNA and proteins) in a sample requires high multiplexity and large dynamic range not available in currently available technologies.

In addition, current methods often rely on the use of enzymatic reactions such as ligation and PCR amplification. The requirement for such enzymes can increase the cost and complexity of current detection methods. Additionally, the requirement for an enzymatic reaction may introduce errors or may limit the range of analytes that can be detected, or require conditions that degrade the target analyte.

Therefore, methods and systems are needed for analyte analysis that allows for detection of target analytes with small sample volume, high multiplexity, reduced assay complexity, a large dynamic range and the ability to detect multiple different species of target analytes, including proteins, nucleic acids, and complexes in a single assay. These assays should be capable of being performed with high sensitivity and specificity.

### SUMMARY OF THE INVENTION

According to some embodiments, provided herein is a method for identifying a presence or absence of one or more distinct target analytes in a sample. In some embodiments, the method comprises: distributing a sample suspected of comprising N distinct target analytes on a substrate such that the target analytes, if present, bind to the substrate at spatially separate regions.

In some embodiments, the method also comprises contacting said sample with N distinct binding probe pairs, wherein each of said N distinct binding probe pairs comprises a first target binding probe and a second target binding probe, wherein said first target binding probe comprises a first specificity determining oligonucleotide, and wherein said second target binding probe comprises a second specificity determining oligonucleotide, wherein said first and second target binding probes are configured to selectively bind as a pair to one of said N distinct target analytes.

In some embodiments, the method also comprises performing M cycles of analyte detection, wherein M is greater than 1, thereby generating a signal detection sequence from one or more of said spatially separate regions, wherein said signal detection sequence comprises redundant data for error correction, each cycle comprising: contacting said sample with an ordered detection probe reagent set comprising X distinct bridging probes each comprising a detectable marker, a first bridging probe oligonucleotide complementary to said first specificity determining oligonucleotide of at least one of said N distinct binding probe pairs, and a second bridging probe oligonucleotide complementary to said second specificity determining oligonucleotide of said at least one of said N distinct binding probe pairs; washing said substrate to remove said bridging probes that are not bound to one of said N distinct binding probe pairs; detecting a presence or absence of a signal from said detectable marker at the spatially separate regions; and if another cycle is to be performed, exposing said substrate to conditions capable of removing said bridging probe from said target analytes.

In some embodiments, the method also comprises analyzing the signal detection sequence to identify the presence or absence of the one or more distinct target analytes in said sample.

In some embodiments, the signal detection sequence from said spatially separate region comprises a signal from at least two distinct detectable markers. In some embodiments, the signal detection sequence comprises one or more cycles with no detectable marker from said spatially separate region.

In some embodiments, said redundant data in said signal detection sequence comprises at least 2 cycles, 3 cycles, 4 cycles, 5 cycles, 10 cycles, 15 cycles, or 20 cycles of analyte detection.

In some embodiments, performing said M cycles of analyte detection generates at least K bits of information per cycle for said N distinct target analytes, wherein said at least K bits of information are used to determine L total bits of information, wherein K×M=L bits of information and L>log 2 (N), and wherein said L bits of information are used to determine the presence or absence of said N distinct target analytes. In one embodiment, K=log 2(X). In one embodiment, X<N. In one embodiment, X=N. In certain embodiments, N is 10 or more, 20 or more, 30 or more, 40 or more, 50 or more, 60 or more, 70 or more, 80 or more, 90 or more, or 100 or more.

In some embodiments, said first and second bridging probe oligonucleotides comprise DNA, RNA, PNA, or LNA. In some embodiments, said first and second specificity determining oligonucleotides comprise, DNA, RNA, PNA, or LNA.

In some embodiments, distributing said sample on said substrate is performed before contacting said sample with said N distinct binding probe pairs. In other embodiments, distributing said sample on said substrate is performed after contacting said sample with said N distinct binding probe pairs. In some embodiments, distributing said sample on said substrate is performed before contacting said sample with said ordered detection probe reagent during the initial cycle.

In some embodiments, said sample is a specimen, a culture, a lysate, a supernatant or a collection from a biological material. In certain embodiments, said sample comprises cell extracts or body fluids. In certain embodiments, said sample comprises immunoprecipitated proteins. In other embodiments, said sample comprises extracts from animal, plant or microbial organisms. In certain embodiments, said sample comprises toxins, allergens, hormones, steroids, or cytokines.

In one embodiment, said sample comprises modified proteins. In specific embodiments, said modified proteins are methylated, phosphorylated, or acetylated.

In another embodiment, said sample comprises one or more immuno-precipitated protein complexes.

In some embodiments, said one or more distinct target analytes comprise a polypeptide. In specific embodiments, said polypeptide is a single protein or a protein complex.

In some embodiments, said one or more distinct target analytes is a polynucleotide. In other embodiments, said one or more distinct analytes are toxins, allergens, hormones, steroids, or cytokines.

In some embodiments, at least one of said N distinct target analytes is a single molecule. In another embodiment, at least one of said N distinct target analytes is a protein-protein or protein-nucleic acid complex. In specific embodiments, said complex is cross-linked with reversible or irreversible linkers.

In some embodiments, said substrate is in the form of a slide, a plate, a chip, or a bead.

In some embodiments, said first target binding probe and/or said second target binding probe comprises an antibody, an aptamer or a complementary oligonucleotide sequence capable of binding to the target analyte. In some embodiments, said first and second target binding probes of one of said X distinct binding probe pairs are configured to selectively bind to different locations on the target analyte associated with said binding probe pair.

In some embodiments, said first and second specificity determining oligonucleotides are at least 12 bp, 13 bp, 14 bp, 15 bp, 16 bp, 17 bp, 18 bp, 19 bp, or 20 bp in length.

In some embodiments, contacting said sample with said N distinct binding probe pairs comprises providing conditions sufficient for binding of the first and second target binding probes to the one or more distinct target analytes.

In some embodiments, said first and second bridging probe oligonucleotides are part of a contiguous oligonucleotide sequence. In certain embodiments, said first and second bridging probe oligonucleotides are at least 12 bp, 13 bp, 14 bp, 15 bp, 16 bp, 17 bp, 18 bp, 19 bp, or 20 bp in length.

In some embodiments, the detectable marker is a fluorophore. In other embodiments, said detectable marker is capable of generating a fluorescent, chemiluminescent, or electrical signal when said bridging probe is bound to said binding probe. In certain embodiments, the detectable marker comprises a nucleic acid tail region comprising a homopolymeric base region of at least 20 bp, 30 bp, 40 bp, 50 bp, 60 bp, 70 bp, 80 bp, 90 bp, or 100 bp in length.

In some embodiments, contacting said sample with said ordered detection probe reagent set comprises providing conditions sufficient for hybridizing the first and second specificity determining oligonucleotides with their respective first and second bridging probe oligonucleotides.

In certain embodiments, said signal, if present, is generated by a single detectable marker.

In some embodiments, said ordered detection probe reagent set for at least two of said M cycles are distinct from each other.

In some embodiments, detecting the presence or absence of the signal comprises optically scanning said substrate for a signal from said detectable marker at said spatially separate regions. In other embodiments, detecting the presence or absence of the signal comprises measuring an electrical signal generated by said detectable marker.

In some embodiments, removing said bridging probe comprises separating the first and second specificity determining oligonucleotides from their respective first and second bridging probe oligonucleotides. In specific embodiments, said separation comprises denaturing the sample. In certain embodiments, said denaturing comprises heat, denaturation agents, salts, or detergents. In other embodiments, removing said bridging probe comprises separating said first and second target binding probes from said one or more distinct target analytes.

In some embodiments, said first and second bridging probe oligonucleotides are not exposed to a polymerase amplification reaction. In other embodiments, said first and second specificity determining oligonucleotides are not exposed to a polymerase amplification reaction. In certain embodiments, said first and second specificity determining oligonucleotides are not exposed to an enzymatic ligation reaction.

Also provided herein, according to some embodiments, is a method for identifying a presence or absence of one or more distinct target analytes in a sample. In some embodiments, the method comprises contacting a sample suspected of comprising N distinct target analytes with N distinct binding probe pairs, wherein each of said N distinct binding probe pairs comprises a first target binding probe and a second target binding probe, wherein said first target binding probe comprises a first specificity determining oligonucleotide, and wherein said second target binding probe comprises a second specificity determining oligonucleotide, wherein said first and second target binding probes are configured to selectively bind as a pair to one of said N distinct target analytes.

In some embodiments, the method also comprises contacting said sample with a detection probe reagent set comprising N distinct bridging probes each comprising a functional substrate binding group, a first bridging probe oligonucleotide complementary to said first specificity determining oligonucleotide of at least one of said N distinct binding probe pairs, and a second bridging probe oligonucleotide complementary to said second specificity determining oligonucleotide of said at least one of said N distinct binding probe pairs. In some embodiments, the method also comprises removing unbound bridging probes from said sample.

In some embodiments, the method also comprises distributing said sample on a substrate such that target-analyte bound bridging probes bind to the surface of said substrate via said functional substrate binding group at spatially separate regions of said substrate.

In some embodiments, the method also comprises performing M cycles of analyte detection, wherein M is greater than 1, thereby generating a signal detection sequence from one or more of said spatially separate regions, wherein said signal detection sequence comprises redundant data for error correction, each cycle comprising: contacting said sample with an ordered probe reagent set comprising X distinct probes each comprising a detectable marker and a sequence complementary to one of said N distinct bridging probes; washing said substrate to remove unbound probes; detecting a presence or absence of a signal from said detectable marker at the spatially separate regions; and if another cycle is to be performed, exposing said substrate to conditions capable of removing said bridging probe from said target analytes.

In some embodiments, the method also comprises analyzing the signal detection sequence to identify the presence or absence of the one or more distinct target analytes in said sample.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead placed upon illustrating the principles of various embodiments of the invention.

FIG. 1 illustrates an embodiment of a complex formed using a pair of target binding probes and a bridging oligonucleotide to detect a single target analyte bound to the surface of the substrate, according to an embodiment of the invention.

FIG. 2 illustrates a flow chart for cycled detection of an analyte bound to a pair of binding probes, according to an embodiment of the invention.

FIG. 3 provides a flow chart for sample preparation to detect protein-protein or protein-nucleic acid complexes, according to some embodiments of the invention.

FIG. 4 is a diagram of a substrate comprising target analytes (e.g., proteins, DNA, RNA, and complexes thereof) from a sample bound to the substrate at spatially separate regions, according to an embodiment of the invention.

FIG. 5 is a top view of a solid substrate with analytes (i.e., analytes A, B, C, and D) randomly bound to the substrate, according to one embodiment of the invention.

### DETAILED DESCRIPTION

The figures and the following description relate to various embodiments of the invention by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### Definitions

“Sample” as used herein includes a specimen, culture, lysate, supernatant or collection from a biological material. Samples may be derived from or taken from a mammal, including, but not limited to, humans, monkey, rat, or mice. Samples may also be derived from plant or microbial organisms. A sample may be an immunoprecipitation of a specimen, culture, lysate, supernatant or collection from a biological material. Samples may include materials such as, but not limited to, cultures, blood, tissue, formalin-fixed paraffin embedded (FFPE) tissue, saliva, hair, feces, urine, and the like. These examples are not to be construed as limiting the sample types applicable to the present invention.

The term “substrate” as used herein refers to any solid or semi-solid support used for adhering to analytes (i.e., nucleic acids or proteins) of interest. A substrate can be made of any suitable material, such as, but not limited to, glass, metal, plastic, membranes, a gel, silicon, carbohydrate surfaces, etc. Substrates can be made of a material that facilitates binding through non-covalent interactions, such as polystyrene. A substrate can be flat two-dimensional surfaces or three-dimensional surfaces, such as micro-beads or micro-spheres. Substrates can be coated or treated with substances to alter the binding characteristics of the substrate to analytes of interest (e.g., glass or silicon surfaces treated with amino silane and glass surfaces treated with epoxy silane-derivatized or isothiocyanate). Substrates may also be coated or bound to adapters (such as antibodies or oligonucleotides) that specifically bind targets of interest. Adapters, including antibodies or oligonucleotide adapters coated on substrates, can be used to generate addressable arrays wherein the location of the oligonucleotide adapters at distinct regions on the substrate correspond to specific targets.

A “target analyte” or “analyte” refers to a molecule, compound, complex substance or component that is to be identified, quantified, and otherwise characterized. A target analyte can be, but not limited to a polypeptide, a lipid, a toxin, a hormone, an allergen, a protein (folded or unfolded), a protein isoform, an oligonucleotide molecule (RNA, cDNA, or DNA), a fragment thereof, a modified molecule thereof, such as a modified nucleic acid, or a combination thereof, e.g., a complex formed from a combination thereof. Generally, a target analyte can be at any of a wide range of concentrations, in any volume of solution (e.g., as low as the picoliter range). For example, samples of blood, serum, formalin-fixed paraffin embedded (FFPE) tissue, saliva, urine, or lysates derived from animal, plant, or microbial sources could contain various target analytes. The target analytes are recognized by target binding probe pairs, which are used in conjunction with bridging probes to identify and quantify the target analytes using electrical or optical detection methods.

Modifications to a target protein, for example, can include post-translational modifications, such as attaching to a protein other biochemical functional groups (such as acetate, phosphate, various lipids and carbohydrates), changing the chemical nature of an amino acid (e.g. citrullination), or making structural changes (e.g. formation of disulfide bridges). Examples of post-translational modifications also include, but are not limited to, addition of hydrophobic groups for membrane localization (e.g., myristoylation, palmitoylation), addition of cofactors for enhanced enzymatic activity (e.g., lipolyation), modifications of translation factors (e.g., diphthamide formation), addition of chemical groups (e.g., acylation, alkylation, amide bond formation, glycosylation, oxidation), sugar modifications (glycation), addition of other proteins or peptides (ubiquination), or changes to the chemical nature of amino acids (e.g., deamidation, carbamylation).

In other embodiments, target analytes are oligonucleotides that have been modified. Examples of DNA modifications include DNA methylation and histone modification.

The term “complex,” as used herein, refers to a biological entity wherein multiple individual subunits or other components are in close physical association with each other. For example, a protein complex can comprise multiple individual protein subunits. Similarly, a nucleic acid complex, such as a ribosome, can comprise multiple individual nucleic acid subunits. In addition, complexes can be formed between subunits of different compositions, such as protein subunits in association with nucleic acid subunits. In general, a subunit within a complex provides a specific function that is important for the overall function of the complex. In some instances, subunits can improve the function of the complex, while in other instances, subunits can inhibit the function of the complex. In some instances, a subunit can be essential for the overall function of the complex. Complexes, in certain examples, can be composed of a well-defined list of discrete components, such as multi-unit protein enzymes. While in other examples, complexes can refer to association between a defined subunit, or multiple defined subunits, and another general, yet undefined, type of component. For example, a transcription factor can associate with multiple DNA promoter elements that contain a conserved motif, but are not strictly conserved sequences.

In general, complexes can be separated into their individual subunits or other components under appropriate conditions without physical cleavage. In some instances, subunits or other components of a complex can remain associated during standard purification conditions allowing purification of the complete complex. In some instances, the subunits or other components of a complex are not in a strong enough association to remain associated during standard purification conditions. In such instances, the subunits or other components of a complex can be cross-linked to form a stable complex capable of remaining associated throughout purification.

“Cross-linking” refers to the use of chemical agents to form reversible or irreversible linkages between components of a complex when they are in close physical association with each other. Cross-linking can be between two proteins, between two nucleic acids, between a protein and a nucleic acid, or between any two separate entities envisaged by those skilled in the art. In some instances, cross-linking can be reversible, either through use of another chemical agent or by other means known to those skilled in the art.

The term “probe,” (e.g., target binding probe or detection probe) as used herein, refers to a molecule that is capable of binding to other molecules (e.g., oligonucleotides comprising DNA or RNA, polypeptides or full-length proteins, etc.). The target binding probe comprises a structure or component that binds to the target analyte. In some embodiments, multiple target binding probes may recognize different parts of the same target analyte. Examples of target binding probes include, but are not limited to, an aptamer, an antibody, a polypeptide, an oligonucleotide (DNA, RNA), or any combination thereof. In certain aspects, probes comprise a detectable label or tag. In certain aspects, probes are modified for conjugation of a detection moiety or a substrate binding moiety. In certain aspects, oligonucleotide target binding probes are modified with a peptide nucleic acid (PNA) to block binding of a label for optimization of detection methods to account for different binding activities of target binding probe. Target binding probe can have a cross-reactivity with non-target sequences. In certain aspects, target binding probes have a cross-reactivity with non-target sequence variant of greater than 2%, 5%, 10%, 15%, 20%, 25%, 50% or 75%. In general, the affinity of an oligonucleotide probe to a target oligonucleotide sequence increases continuously with oligonucleotide length. In a preferred embodiment, oligonucleotide probes have a dissociation constant in the range of about 10⁻⁹ to 10⁻⁶ molar, in the range of 10⁻⁹ to 10⁻⁸ molar, in the range of 10⁻⁸ to 10⁻⁷ or the range of 10⁻⁷ to 10⁻⁶ molar.

“Binding,” as used herein, refers to a specific, targeted interaction between two entities, such as an antibody binding with a desired affinity to an antigen or a nucleic acid probe binding, i.e. base pairing, with a desired melting temperature to a target nucleic acid. The term “binding” is not limited to these examples, and one skilled in the art would be able to recognize other examples of what is an appropriate binding interaction in a given context.

“Hybridizing” as used herein, refers to the annealing of a nucleic acid molecule to another nucleic acid molecule through the formation of one or more hydrogen bonds (e.g., base pairing of complementary nucleotides by hydrogen bond formation). Nucleic acids may be hybridized under any conditions known and used in the art to efficiently anneal oligonucleotides to nucleic acids of interest. Oligonucleotides may be hybridized in conditions that vary significantly in stringency to compensate for binding activity with respect to target binding and off-target binding.

In embodiments wherein the target binding probe is an oligonucleotide, the affinity of an oligonucleotide target binding probe to a target oligonucleotide sequence, in general, increases continuously with oligonucleotide length. In a preferred embodiment, oligonucleotide target binding probes have a dissociation constant in the range of about 10⁻⁹ to 10⁻⁶ molar, in the range of 10⁻⁹ to 10⁻⁸ molar, in the range of 10⁻⁸ to 10⁻⁷ or the range of 10⁻⁷ to 10⁻⁶ molar.

Methods to determine specific or preferential binding are well known in the art. A molecule exhibits “specific binding” or “preferential binding” if it reacts or associates more frequently, more rapidly, with greater duration and/or with greater affinity with a particular cell or substance than it does with alternative cells or substances. For example, an antibody “specifically binds” or “preferentially binds” to a target if it binds with greater affinity, avidity, more readily, and/or with greater duration than it binds to other substances. For example, an antibody that specifically or preferentially binds to a conformational epitope of a protein target biomolecule is an antibody that binds this epitope with greater affinity, avidity, more readily, and/or with greater duration than it binds to other epitopes on the same target biomolecule or epitopes on different target biomolecules. It is also understood by reading this definition that, for example, an antibody (or moiety or epitope) that specifically or preferentially binds to a first target biomolecule may or may not specifically or preferentially bind to a second target biomolecule. As such, “binding”, “specific binding” or “preferential binding” does not necessarily require (although it can include) exclusive binding.

“Detectable marker” as used herein, refers to a molecule capable of producing a signal for detecting a target biomolecule. The marker can be, but is not limited to, a fluorescent marker. The marker can comprise, but is not limited to, a fluorescent molecule, chemiluminescent molecule, chromophore, enzyme, enzyme substrate, enzyme cofactor, enzyme inhibitor, dye, metal ion, metal sol, ligand (e.g., biotin, avidin, streptavidin or haptens), radioactive isotope, markers for electrical detection (e.g., ISFET detection), markers that produce a change in pH upon a subsequent reaction, and the like. A detectable marker may comprise a plurality or a combination of markers.

“Detection” as used herein, refers to the identification of a signal produced by the methods described herein. “Detection” may or may not comprise one or more analysis steps. “Detection” as used herein, may comprise performing any method known to one of ordinary skill in the art to identify the target molecule from the signal produced by the methods described herein. For example, in certain embodiments, “detection” may comprise use of sequencing methods known in the art and/or microscopy or other imaging methods. “Detection” includes optical detection or electrical detection.

The term, “complementary” as used herein refers to a complement of the sequence by Watson-Crick base pairing, whereby guanine (G) pairs with cytosine (C), and adenine (A) pairs with either uracil (U) or thymine (T). A sequence may be complementary to the entire length of another sequence, or it may be complementary to a specified portion or length of another sequence. One of skill in the art will recognize that U may be present in RNA, and that T may be present in DNA. Therefore, an A within either of a RNA or DNA sequence may pair with a U in a RNA sequence or T in a DNA sequence. The term “complementary” is used to indicate a sufficient degree of complementarity or precise pairing such that stable and specific binding occurs between nucleic acid sequences e.g., between a homology region of the detection probe and the specificity determining oligonucleotide of interest. It is understood that the sequence of a nucleic acid need not be 100% complementary to that of its target or complement. In some cases, the sequence is complementary to the other sequence with the exception of 1-2 mismatches. In some cases, the sequences are complementary except for 1 mismatch. In some cases, the sequences are complementary except for 2 mismatches. In other cases, the sequences are complementary except for 3 mismatches. In yet other cases, the sequences are complementary except for 4, 5, 6, 7, 8, 9 or more mismatches.

A “cycle” is defined by completion of one or more passes and stripping of the probes from the target analyte. Subsequent cycles of one or more passes per cycle can be performed. Multiple cycles can be performed on a single target analyte or sample. For proteins, multiple cycles will require that the probe removal (stripping) conditions either maintain proteins folded in their proper configuration, or that the probes used are chosen to bind to peptide sequences so that the binding efficiency is independent of the protein fold configuration.

“Bit” as used herein refers to a basic unit of information in computing and digital communications. A bit can have only one of two values. The most common representations of these values are 0 and 1. The term bit is a contraction of binary digit. In one example, a system that uses 4 bits of information can create 16 different values. All single digit hexadecimal numbers can be written with 4 bits. Binary-coded decimal is a digital encoding method for numbers using decimal notation, with each decimal digit represented by four bits. In another example, a calculation using 8 bits, there are 2⁸ (or 256) possible values.

Abbreviations used in this application include the following: “DNA” (deoxyribonucleic acid), “RNA” (ribonucleic acid) and “ISFET” (ion-sensitive field-effect transistor).

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### Overview

Detection techniques that can be used for highly multiplexed single molecule identification and quantification of analytes using proximity binding detection are described herein. Using these techniques, one can perform detection and quantification with high sensitivity and specificity.

In some embodiments, provided herein is a method of detecting one or more target analytes by binding two target probes to separate epitopes on the target analyte, then detecting the proximity of both probes through a secondary bridging probe, which binds to both target probes simultaneously. The presence or absence of this binding interaction for a single analyte can then be probed. This can be done, for example, to facilitate detection of the presence or absence of the target analyte, a modification of the target analyte, or the presence of one or more entities in a target analyte complex.

Similar procedures often involve a further enzymatic amplification step, such as ligation of oligos on the two target probes in proximity, and/or amplification to generate a signal. In contrast, the methods and systems provided herein are based on direct detection of one or more detectable markers that are part of the bridging probe, which itself preferentially binds to the complex only when both target probes are present in the appropriate proximity (i.e., are bound to the target epitopes of the target analyte). Thus, no subsequent ligation or amplification steps are needed. Due to the stochastic nature of single molecule binding interactions, single molecule detection is subject to false negatives (e.g., where no probe is bound to a target analyte), and false positives (e.g., where an incorrect probe is bound to a target analyte or other entity after a washing step). Thus, provided herein are methods of performing the proximity binding assay to detect the target analyte using cycled detection to reduce errors of detection. The cycled detection generates a signal sequence which can then be matched to a code specific for a target analyte. The signal sequence includes redundant data to allow for the signal sequence to be matched to a target analyte code despite the presence of false positives or false negatives from individual cycles. In other words, performing multiple cycles of the proximity binding assay allows for generating a signal sequence that includes redundant data (also referred to as “parity data”). Performing multiple cycles sufficient to generate such redundant data allows for data refinement (e.g., error correction) and/or data validation using an error-correcting code (also referred to as an error-correcting scheme or error correction code).

The detection of targets and their authentication based on repeat hybridizations enables highly multiplexed and accurate single target analyte identification and counting for quantification. Relative and absolute abundance can also be quantified.

Proximity Binding Cycled Detection of Target Analytes

A general process for identifying target analytes immobilized on a substrate using proximity binding detection is described below. Sample is distributed onto a substrate where single analytes bind at spatially separated areas on the substrate. Then the sample is exposed to i) paired target probes, and ii) cycles of ordered probe sets comprising bridging probes with a detectable marker to generate a detection sequence. In some embodiments, the cycled detection can also be done with the paired target probes, e.g., to generate additional information based on more than two epitopes present on an analyte.

Proximity Binding Detection of Target Analytes—Generalized

A sample comprising target analyte is immobilized onto the surface of a solid substrate, such that individual target analytes are bound at spatially separate areas of the substrate. Pairs of target binding probes that bind specifically to epitopes on the target analytes are flowed over the immobilized sample. Target binding probes are paired to form a distinct pair of target binding probes, each pair specifically recognizing a target analyte. Target binding probes include, but are not limited to, antibodies, aptamers, and nucleic acid probes. Conditions are provided to optimize target binding probe recognition of its target analyte, such as conditions for optimal antibody binding or optimal nucleic acid probe hybridization. In some embodiments, conditions can be provided to facilitate binding of two types of target binding probes, such as an antibody paired with a nucleic acid probe.

Following binding of the target binding probes, the substrate and sample is washed to remove non-specifically bound target binding probes. In some embodiments, the wash conditions are known to those skilled in the art, and may include a variety of temperatures, salt compositions and concentrations, and/or detergent compositions and concentrations. In some embodiments, the wash conditions are designed to maximize removal of non-specific binding. In other embodiments, the wash conditions take into consideration maintaining complexes or the native conformation of molecules. In some embodiments, the wash conditions take into consideration if two types of target binding probes are used, for example, an antibody used in conjunction with a nucleic acid probe.

In preferred embodiments, the target binding probe comprises a target binding entity bound to a specificity determining oligonucleotide. Each specificity determining oligonucleotide is engineered, using bioinformatic computational methods well known to those skilled in the art, to have a melting temperature (Tm) within a narrow range such that all specificity determining oligonucleotides possess a similar Tm. The specificity determining oligonucleotides are also engineered, using similar bioinformatic computational methods, to avoid sequence similarity to other specificity determining oligonucleotides to reduce non-specific hybridization to incorrect bridging probes, as discussed further below. Covalent attachment of oligonucleotides to target binding probes is well known in the art, see for example Liu et al. (BioProcess International; 10(2) February 2012), which is incorporated herein by reference in its entirety. In some embodiments, a specificity determining oligonucleotide comprises only a nucleotide sequence engineered to hybridize to a bridge probe. In some embodiments, a specificity determining oligonucleotide comprises nucleotide sequences in addition to the nucleotide sequence engineered to hybridize to a bridge probe, e.g., a polynucleotide linker including, but not limited to, a polynucleotide linker used for covalently attaching a target binding probe and/or a polynucleotide linker that reduces a target binding probe sterically hindering hybridization between a specificity determining oligonucleotide and a bridge probe. In some embodiments, wherein a target binding probe is a polynucleotide, a specificity determining oligonucleotide and the target binding probe are part of a single contiguous polynucleotide, optionally comprising a polynucleotide linker separating the specificity determining oligonucleotide and the target binding probe. Furthermore, the size of the specificity determining oligonucleotide, the polynucleotide linker, or the specificity determining oligonucleotide and the polynucleotide linker is such that a bridge probe can only hybridize to two specificity determining oligonucleotides if both oligonucleotides are in sufficient proximity to each other, e.g. if both specificity determining oligonucleotides are associated with same target analyte at their target epitopes.

Following target binding probes binding, bridging probes are flowed over the immobilized sample under conditions that facilitate hybridization, i.e. base pairing, between the bridging probe and the specificity determining oligonucleotides covalently attached to the target binding probes. The sample is then washed to remove non-specific hybridization. For example, the sample is washed at a temperature such that a bridging probe will only remain bound when base paired to both specificity determining oligonucleotides bound to a target analyte. In one embodiment, this temperature is above the Tm range used to design the specificity determining oligonucleotides. In some embodiments, the wash conditions are designed to maximize removal of non-specific binding. In other embodiments, the wash conditions take into consideration maintaining complexes or the native conformation of molecules. In some embodiments, the wash conditions take into consideration avoiding removal of the specifically bound target binding probes. In some embodiments, the wash conditions take into consideration if two types of target binding probes are used, for example, an antibody used in conjunction with a nucleic acid probe.

In certain embodiments, the proximity binding detection assay comprises performing at least N detection cycles to generate a target identification signal detection sequence for at least one of the spatially separate regions on the substrate. In certain embodiments, N is at least two, and each cycle comprises contacting the substrate comprising the immobilized target analytes with ordered detection probe reagent set comprising Y distinct bridging probes. The ordered detection probe reagent set comprises a plurality of bridging probes that each directly or indirectly bind preferentially to at least one of the one or more target biomolecules, preferably via binding to two target binding probes in proximity. The plurality of bridging probes each comprise a target identification detectable marker. The proximity binding detection assay further comprises the step of removing unbound bridging probes from the surface of the substrate; detecting the presence or absence of a signal from the detectable marker at the spatially separate regions; and if the cycle number is less than N, removing bound target detection probes from the substrate.

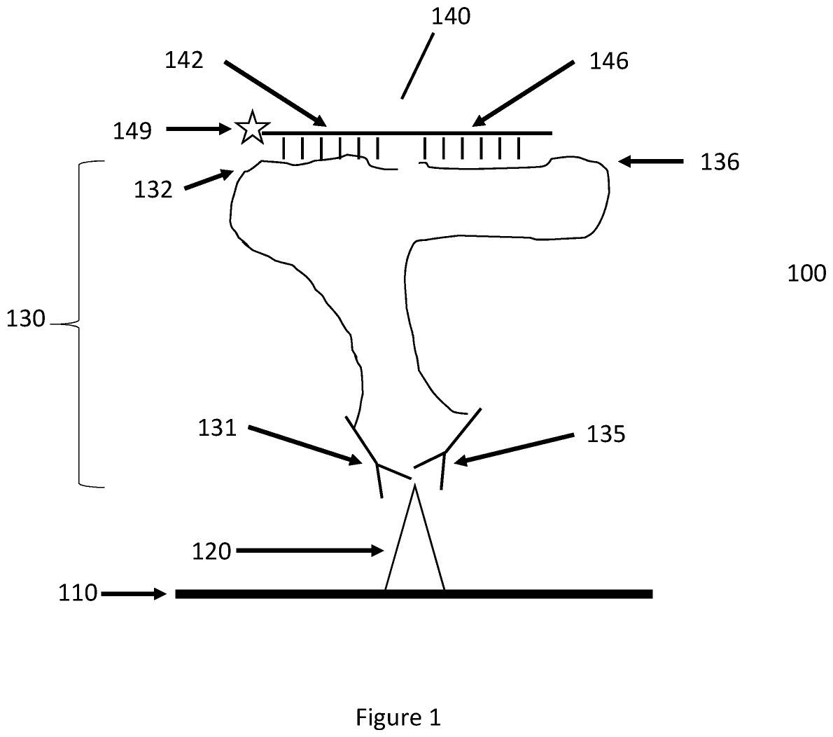

A diagram of a complex 100 formed during a proximity binding detection of a single analyte, according to some embodiments, is shown in FIG. 1. A target analyte 120 is immobilized on a solid substrate support 110. A set of binding probe pairs are than added to the substrate to bind specifically to their respective target analytes. A binding probe pair 130 includes a first binding probe 131 and a second binding probe 135 that each bind to the respective target analyte 120 at different epitopes. Thus, the binding probe pair 130 is held in close proximity due to being bound to the same target analyte 120 immobilized on the surface of the substrate 110. Each binding probe has a specificity determining oligonucleotide (i.e., the first binding probe 131 has a first specificity determining oligonucleotide 132, and the second binding probe 135 has a second specificity determining oligonucleotide 136).

When in close proximity, the first and second specificity determining oligonucleotides are complementary to oligonucleotide sequences on a bridging probe 140. The bridging probe 140 comprising a detectable marker 149, a first bridging probe oligonucleotide 142 complementary to the first specificity determining oligonucleotide 132, and a second bridging probe oligonucleotide 146 complementary to the second specificity determining oligonucleotide 136.

Thus, when a bridging probe 140 is added to the surface of the substrate 110, the bridging probe 140 will bind to target analytes that are bound to their respective binding probe pair 130. After removal of unbound probes, a signal generated by the detectable marker 149 of the bound bridging probe 140 can be detected and provide information about the identity of the complex on the substrate.

Several elements within the proximity binding assay are engineered to achieve specific labeling of the target analyte. The cooperative binding facilitated by the distinct binding probe pair provides an important discrimination step that achieves the increased accuracy and specificity of analyte detection of the method described herein. The proximity binding detection method is engineered such that a single target binding probe is not sufficient to achieve proper labeling of the target analyte. Instead, the distinct binding probe pair, when both are bound to the same target analyte, work together to achieve the specific labeling by the bridging probe. This can be achieved by exposing bridging probe sets on the surface of the substrate to washing conditions that selectively remove unbound and singly-bound probes, while minimizing perturbation of bridging probes bound to both target binding probes of the target binding probe pair.

As described above, the distinct binding probe pair works cooperatively to specifically label the analyte. To do so, attached to each target binding probe is a unique, specificity determining oligonucleotide specific to each target binding probe. In turn, the specificity determining oligonucleotides are engineered to hybridize through complementary base pairing to a portion of a specific bridging probe. The two complementary regions 142 and 146 on each bridging probe are engineered to specifically hybridize to distinct specificity determining oligonucleotides. Furthermore, the size of the bridging probe is such that it can only hybridize to two specificity determining oligonucleotides if both oligonucleotides are in sufficient proximity to each other, e.g. if both specificity determining oligonucleotides are associated with same target analyte at their target epitopes.

Following appropriate wash conditions, bridging probes will preferentially remain bound when both complementary regions of the bridging probe are properly hybridized to two specificity determining oligonucleotides, i.e. when the distinct binding probe pair cooperatively facilitates labeling of the target analyte. Thus, multiple layers of specificity are engineered into the proximity binding detection method to provide a key discrimination step to achieve improved accuracy and specificity in analyte detection. Following the labeling steps in the proximity binding detection method described above, the bridging probe is detected to accurately and specifically identify and quantify the target analyte.

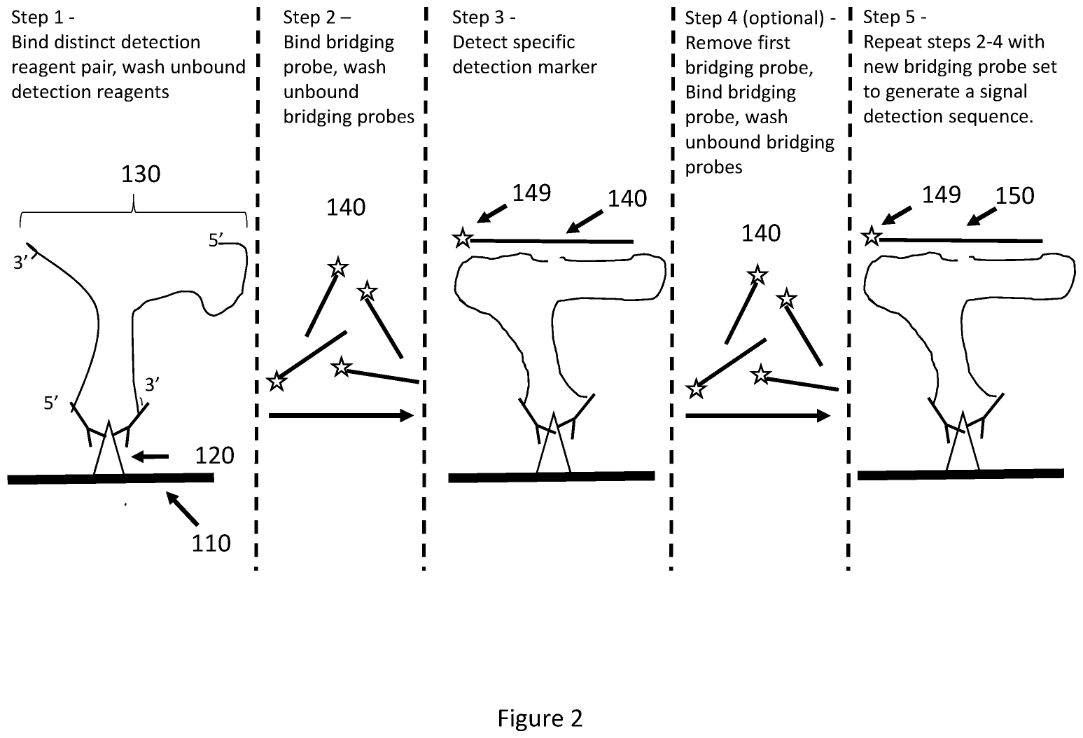

In some embodiments, an analyte detection using target binding probe pairs and a bridging oligo comprising a detectable marker proceeds as illustrated in FIG. 2. A sample is obtained that is suspected of containing at least one analyte of interest 120, although the assay may be used to detect thousands of analytes of interest. The protein of interest is immobilized onto the surface of a substrate 110. In Step 1, a target binding probe pair 130 is added that specifically binds to epitopes on the target analyte. In this embodiment, the target binding probe pair 130 each comprise an antibody specific for a distinct epitope on the target analyte. Each probe comprises a specificity determining oligonucleotide bound to the antibody. Unbound target binding probe pairs are removed by washing.

In Step 2, bridging probes comprising detectable markers are added to the surface of the substrate. In the embodiment shown, the detectable marker is a fluorophore with a specific color associated with each target. These bridging probes bind to the pair of target binding probes when the probes are in sufficient proximity by virtue of their attachment to the target analyte. Specifically, the first bridging probe oligonucleotide of the bridging probe binds to the first specificity determining oligonucleotide of the first target binding probe, and the second bridging probe oligonucleotide of the bridging probe binds to the second specificity determining oligonucleotide of the second target binding probe. After binding, unbound bridging probes are removed by washing under conditions that preferentially removes unbound and singly bound bridging probes, while retaining bridging probes bound to two target binding probes.

In Step 3, the presence or absence, and identity if present, of a fluorophore from the spatially separate region on the substrate comprising the analyte is detected. This signal, or absence thereof, generates a unit of information to be included in a sequential code (i.e., a signal detection sequence) used for identification of the target analyte, or for characterizing the target analyte.

Thus, in order to perform successive rounds of probe binding and detection with other ordered bridging probe sets, in Step 4, the bridging probe bound to the target binding probe pair is removed from the surface by washing under appropriate conditions. These conditions can be selected to only remove the bridging probe, or can include conditions to also remove the first and second target binding probes, such that binding of the same or other variations of target binding probe pairs can also be performed in subsequent detection cycles.

After washing, Steps 2-4 are performed in cycles of detection to generate the signal detection sequence that is used to determine an identity or characteristic of a target analyte. Bridging probes to the same target analyte can have different detectable markers (e.g., different fluorophore emission spectrum) to generate the unique signal detection sequence associated with a target analyte or a characteristic (e.g., modification) of the target analyte. In some embodiments, Steps 1-4 are performed in one or more cycles to allow re-binding of the same or different target binding probes. This can be used, for example, to detect the presence or absence of more than 2 epitopes on a target analyte for further characterization of a target analyte.

An outline of steps performed, according to an embodiment of the invention, is as follows:

1. Flow sample onto a substrate to bind target analytes at spatially separate regions on the substrate.

2. Add a solution comprising a set of target binding probe pairs for each target analyte of interest under conditions that promote binding of the target binding probe to its target analyte.

3. Remove unbound binding probe pairs.

4. Add a solution comprising a set of bridging probes for each target analyte of interest under conditions that promote hybridization of complementary oligonucleotide sequences.

5. Remove unbound bridging probes.

6. Detect a signal from a detectable maker (e.g., a fluorophore) on the bridging probe.

7. If a subsequent detection cycle is to be performed, remove bridging oligo.

8. Perform cycled detection by repeating steps 4-7 (and optionally also steps 2-3, where the target binding probes are also removed from the target analyte after detection in the previous cycle)

Target Analytes

In some embodiments, target analytes can include, but are not limited to, detection of single molecules, such as a protein, a peptide, a DNA or an RNA molecule, detection of modifications to a target analyte, and/or detection of complexes formed between two or more single molecules, with and without modifications.

The above described proximity binding detection technique can be applied to detection of single molecules. Most technologies currently rely on a single target binding probe that recognizes a single molecule. However, reliance on a single target binding probe can lead to inaccurate results, for example if the single target binding probe binds non-specifically to non-targets. The proximity binding detection method improves accuracy through the cooperative binding steps provided by the distinct binding probe pair, as discussed above. In an example, the single molecule is immobilized on a solid substrate support and a distinct binding probe pair specific for the single molecule is provided. Then, a specific bridging probe with a detectable marker is provided that binds the distinct binding probe pair through cooperative binding. Next, the detectable marker is used to accurately quantify and identify the single molecule. Importantly, the method's use of two target binding probes that both bind a single molecule reduces the error generated by either target binding probe alone binding to a target analyte.

In another embodiment, multiple target binding probes can be used to characterize target analytes, such as to determine whether a target analyte is modified or unmodified. For example, a combination of antibodies may be used wherein one antibody is specific for the target analyte, such as a protein of interest, while a second antibody is specific for a broader characteristic, such as a post-translational modification. In this example, analytes of interest with the specific characteristic can be distinguished from analytes of interest without the specific characteristic.

In an illustrative example, detection of whether selected proteins are phosphorylated can be addressed by the present invention. Using conventional techniques, antibodies that distinguish between a phosphorylated and a non-phosphorylated target protein are limited. However, using the proximity binding method, an antibody specific for the protein of interest can be combined with an antibody specific for an amino acid or polypeptide phosphorylation, such as a phosphor-tyrosine or phosphor-serine antibody. Thus, only proteins bound to both antibodies will bind to the bridging probe and generate a detection signal. Thus, phosphorylated proteins of interest can be accurately identified and quantified by the methods provided herein.

Complexes are composed of multiple subunits or other components that associate with each other. In one embodiment of the proximity binding detection method, complexes can be interrogated to identify, characterize and quantify target complexes. The wide range of possible biological complexes that can be interrogated using this method will be appreciated by one skilled in the art and includes, but is not limited to, protein-protein complexes. In some embodiments, the complex is a multi-unit enzyme, a nucleic acid complex, a ribosome, DNA bound to nucleic acid binding proteins such a transcription factors, or a receptor-ligand pair.

In general, the association of subunits or other components within a complex facilitates the performance of a biological function by the complex. However, the exact composition of subunits or other components within a complex is frequently not static. For example, the activity of a complex may be regulated through control of the exact subunit composition. In some instances, a complex is not active until all subunits are present. Thus, the activity of the complex can be regulated by the availability of subunits. In other instances, a subunit, when present, may act as an inhibitor of a complex's activity. In another embodiment, formation of particular complexes can be used as a proxy for the state of a cell or organism. For example, the formation of signaling complexes can be used a read out for signaling activity within a cell. Therefore, interrogation of the subunit composition can illuminate the activation state of a complex or, more generally, the state of a cell or organism.

In some embodiments, provided herein is a method of detecting and/or quantifying complexes using proximity binding. In one embodiment, a complex is immobilized on a solid substrate such that all the subunits or other components of the complex to be interrogated remain associated. A pair of target binding probes can be used in the assay, wherein each reagent is specific to a distinct component within a complex. As discussed previously, a probe labeled with a detectable marker will only remain bound when both target binding probes bind a target analyte. Thus, detection of a complex will only occur when both components are present within the complex, thereby characterizing the composition of the complex.

In one embodiment, a single pair of target binding probes can be used to characterize the complex. For example, one of the target binding probes within the pair can bind a subunit that defines a complex, while a second target binding probe can bind to a regulatory subunit that defines the activation state of the complex.

In another embodiment, multiple rounds of interrogation can be performed to characterize the composition of a complex. For example, a complex with three or more subunits can be interrogated using sequential rounds of the proximity binding detection method, wherein target binding probes to three or more subunits can be used in combination to determine the full composition of the complex. For example, a first round of interrogation may use target binding probes to a first and second subunit. Then, a subsequent round of interrogation may use target binding probes the first subunit and a third unit. Additional rounds can be performed as well, using target binding probes specific for additional subunits or in various iterative combinations. The detection results from the multiple rounds can be combined and used to characterize the complex's composition.

Other examples of biological complexes include instances where a defined complex associates with unknown, undefined, or variable elements. For example, many protein complexes are known that bind nucleic acids. However, the identity of the nucleic acids themselves can be variable. In such instances, and other situations where the exact composition of a complex is unknown, the proximity binding detection method can be used to interrogate the identity of elements associated with a given complex.

In one example, transcription factors are proteins that recognize DNA with conserved motifs. However, in general, not all DNA that contains a given conserved motif is bound by its cognate transcription factor. In one embodiment of the proximity binding detection method, immunoprecipitation of transcription factors of interest associated with nucleic acids can be performed as a first step. Following dissociation of the nucleic acid from the transcription factor, the nucleic acid can be hybridized to a solid support and its identity interrogated using the proximity binding detection method with target binding probes specific to various nucleic acids, as previously discussed. In another embodiment, the transcription factor bound nucleic acid can be hybridized to a solid support, and the identity of the transcription factors interrogated using the proximity binding detection method with target binding probes specific to various transcription factors. In certain embodiments, the transcription factor bound nucleic acid complexes can be cross-linked, and optionally reversed cross-linked.

Sample Preparation

The present invention provides methods for identifying and quantifying a wide range of analytes, from a single analyte up to tens of thousands of analytes simultaneously over many orders of magnitude of dynamic range, while accounting for errors in the detection assay.

In some embodiments, the target analyte to be interrogated is contained in serum from a variety of sources including, but not limited to, blood and other bodily fluids, from which analytes can be collected using methods known to those skilled in the art, for example, serum collection tubes using dotting factors.

In some embodiments, the target analyte to be interrogated is present in cell culture supernatants and collected using methods known to those skilled in the art including, but not limited to, high speed centrifugation, aspiration, transwell plates, filtration etc.

In some embodiments, the target analyte to be interrogated is present in cellular lysates and collected using methods known to those skilled in the art including, but not limited to, sonication, enzymatic lysis, french press, freeze-thaw, dounce homogenization, high speed centrifugation, molecular weight filtration etc. Cellular lysates can be of eukaryotic or prokaryotic origin, cultured cell lines, tissues, isolated primary cells, ex vivo cultured primary cells, or other sources known to those skilled in the art. In some embodiments, lysis can be performed under denaturing conditions, for example, in a reducing environment where intramolecular and intermolecular bonds are disrupted. In other embodiments, lysis can be performed under non-denaturing conditions, wherein the native conformation of an analyte and/or association of subunits or other components within a complex is maintained.

In some embodiments, the target analyte is collected from the environment, such as from water, food, the atmosphere, man made products, natural products etc. Target analytes are collected from the environment by methods known to those skilled in the art.

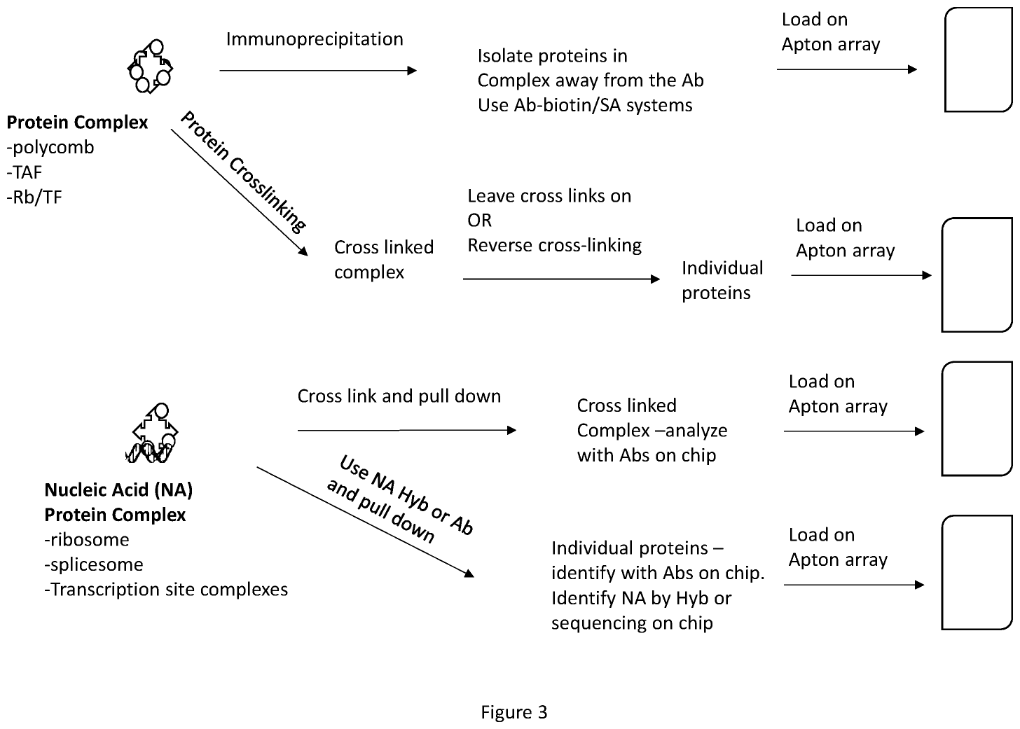

In some embodiments, immunoprecipitation of the target analyte or target complex is performed (see, e.g., FIG. 3). In brief, a sample suspected of containing the target analyte or complex is mixed with an antibody specific for the target analyte or complex under conditions that promote binding of the antibody to its target, such as rotation at 4 degrees. Immunoprecipitation can use either monoclonal or polyclonal antibodies. In some embodiments, the antibody can be specific for an artificial moiety, or tag, that comprises a portion of the target analyte or complex. In some embodiments, a target complex can be cross-linked prior to immunoprecipitation. Various methods for purifying, or precipitating, the antibody bound target are known to those skilled in the art and include, but are not limited to, steps of washing the sample to remove non-specifically bound molecules, purifying the antibody bound targets using common reagents such as Protein-A/G resins including agarose and magnetic beads, and eluting the target analyte or complex through denaturation, glycine elution, peptide elution, or other elution methods known to those skilled in the art.

In one example, complexes may be cross-linked prior to interrogation (see, e.g., FIG. 3). For example, in some instances, the subunits or other components within a complex may not naturally have a strong enough interaction to remain in complex during the proximity binding detection method. Thus, cross-linking can allow full complexes, which otherwise would dissociate, to be still interrogated. In general, cross-linking is carried out using chemical reagents that cause the formation of covalent bonds between subunits or other components of a complex. For example, formaldehyde can be used to cross-link proteins to other proteins or proteins to nucleic acids. Other chemical cross-linkers are known to those skilled in the art and can be selected based on desired criteria including, but not limited to, requirements dictated by specific complexes, toxicity, ease of use, reversibility of cross-links, in vivo applicability, in vitro applicability, and compatibility with downstream applications.

In some embodiments, the complex can be cross-linked prior to immunoprecipitation. The immunoprecipitated complex can then be immobilized on a solid support and interrogated using the proximity binding detection method. In another embodiment, the complex can first be immunoprecipitated, then the subunits or other components subsequently dissociated from each other and immobilized individually on a solid support. In this example, the individual subunits or other components can then be interrogated as separate target analytes using the proximity binding detection method, as previously discussed. In some instances, the complex can first be cross-linked, then immunoprecipitated, and followed by reverse cross-linking and dissociation of the individual subunits or other components. After immobilization to a solid support, the individual subunits or other components can then be interrogated as separate target analytes using the proximity binding detection method, as previously discussed.

Sample Distribution on an Array

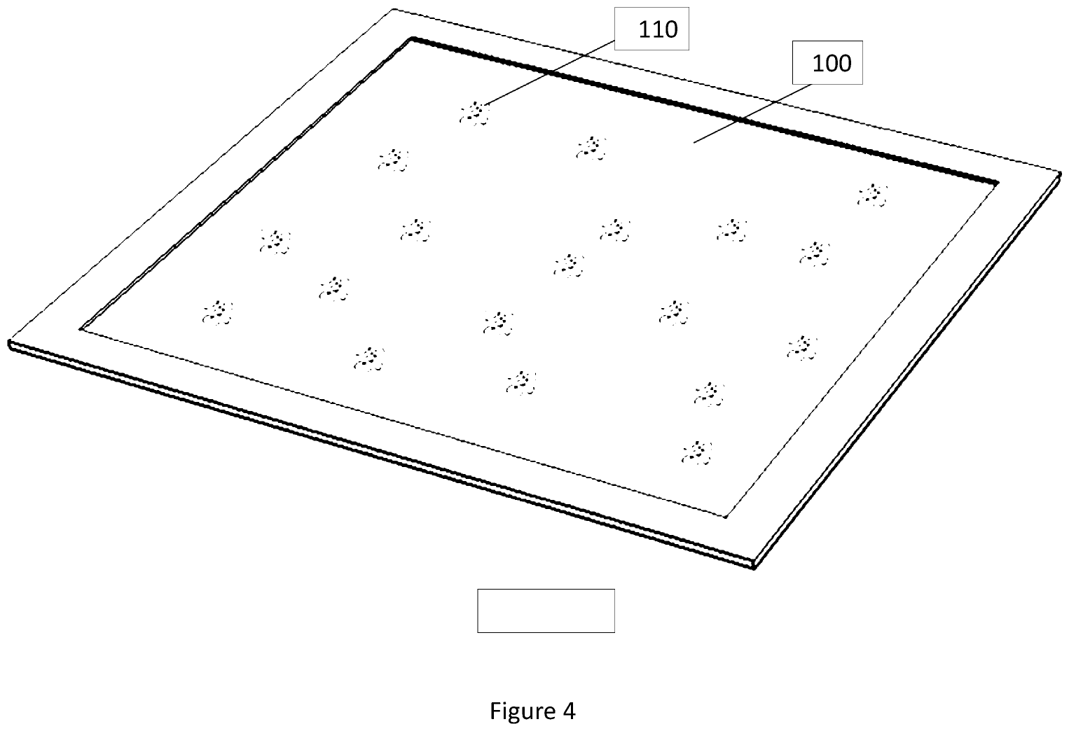

As shown in FIG. 4, a sample comprising analytes 120 (prepared as discussed above) are bound to a solid substrate 110. The substrate 110 can comprise a glass slide, silicon surface, solid membrane, plate, or the like used as a surface for immobilizing the analytes 120. In one embodiment, the substrate comprises a coating that binds the analytes to the surface. In another embodiment, the substrate comprises capture antibodies or beads for binding the analytes to the surface. The analytes can be bound randomly to the substrate and can be spatially separated on the substrate. The sample can be in aqueous solution and washed over the substrate, such that the analytes bind to the substrate. In one embodiment, the proteins in the sample are denatured and/or digested using enzymes before binding to the substrate. In some embodiments, the analytes can be covalently attached to the substrate. In another embodiment, selected labeled probes are randomly bound to the solid substrate, and the analytes are washed across the substrate.

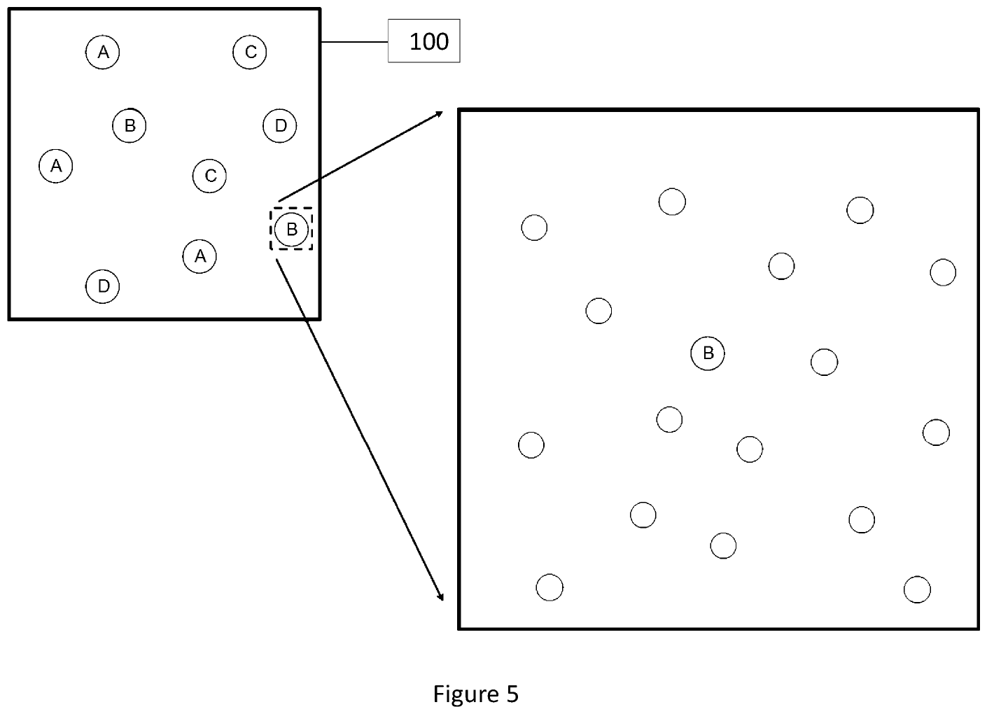

Shown in FIG. 5 is a top view of a solid substrate 110 with analytes randomly bound to the substrate 110. Different analytes are labeled as A, B, C, and D. For optical detection of the analytes, the imaging system requires that the analytes are spatially separated on the solid substrate 110, so that there is no overlap of fluorescent signals.

In some embodiments the solid substrate can be of any composition that facilitates immobilization of target analytes. The solid substrate can comprise a base composition, such as a silicon, glass, synthetic polymer, magnetic, or other material known to those skilled in the art used to immobilize analytes. The solid substrate can be in several shapes or forms, such as beads, slides or wells in a plate. The solid substrate can be further functionalized to facilitate immobilization, such as attachment of reactive chemical groups, antibodies, nucleic acid probes, or other functional groups known to those skilled in the art to immobilize analytes. Immobilization can occur through covalent attachment to the substrate or functional group, non-covalent interactions with the substrate or functional group, targeted binding by antibodies, hybridization to nucleic acid probes, or other interactions known to those skilled in the art to immobilize analytes.

The nature of the substrate binding moieties will correspond to the type of substrate or solid support to be used for binding to the target biomolecule. A substrate can be any solid or semi-solid support used for adhering to analytes/target biomolecules. A substrate can be made of any suitable material, such as, but not limited to, glass, metal, plastic, a gel, membranes, silicon, a carbohydrate surface, etc. Substrate binding moieties can be, for examples, modified nucleotides. Proteins and/or oligonucleotides can be modified by any suitable method known in the art for attachment and/or immobilization of protein and/or nucleic acid to substrates, for example, by conjugation to biotin, generating amine or thiol group modifications, covalent linkage to a thioester or conjugation to a cholesterol-TEG. Modification of oligonucleotides to produce substrate binding moieties may occur at the 5′ terminus, 3′ terminus or at any position within the oligonucleotide. Linkers or spacers may be added between the terminus of the oligonucleotide and the substrate binding moiety. Substrate binding moieties may be bound directly or indirectly to the target biomolecules, probes, tags, agents and oligonucleotides described herein.

The type of solid support chosen will be chosen based on the level of scattering and fluorescence background inherent in the support material and added chemical groups; the chemical stability and complexity of the construct; the amenability to chemical modification or derivatization; surface area; loading capacity and the degree of non-specific binding of the final product. Substrates can be prepared by treating glass or silicon surfaces, for example, with avidin for the binding to biotin-conjugated oligonucleotides. In another example, glass or silicon surfaces can be treated with an amino silane. Oligonucleotides modified with an NH₂ group can be immobilized onto epoxy silane-derivatized or isothiocyanate coated glass slides. Succinylated oligonucleotides can be coupled to aminophenyl- or aminopropyl-derivatized glass slides by peptide bonds, and disulfide-modified oligonucleotides can be immobilized onto a mercaptosilanized glass support by a thiol/disulfide exchange reaction or through chemical cross-linkers. Amine-modified oligonucleotides can be reacted with carboxylate-modified micro-spheres with a carbodiimide, such as EDAC. Substrates may also be magnetic (such as magnetic microspheres) and bind to oligonucleotides conjugated or annealed to magnetic moieties.

Target Binding Probes

As provided herein, a proximity binding assay uses a pair of target binding probes as an intermediate between a target analyte and a bridging probe for target analyte identification or characterization. By requiring the presence of a pair of target binding probes for detection, the incidence of false positive identifications can be decreased, improving the stringency of the assay. In some embodiments, multiple target binding probes can be used to accurately identify specific target analytes when there is no single target binding probe uniquely specific for the target analyte, but the specific target analyte can be distinguished by a combination of characteristics.

In some embodiments, the target binding probes include, but are not limited to, antibodies, aptamers, and nucleic acid probes. Binding to the target analyte is contemplated here to mean how one skilled in the art would envisage binding to occur to a target analyte using target binding probes, such as an antibody binding with a desired affinity to an antigen or a nucleic acid probe binding, i.e. base pairing, with a desired melting temperature to a target nucleic acid.

In some embodiments, the target binding probe binds a protein. In some embodiments, the target binding probe binds nucleic acid. In an embodiment, the target binding probe binds DNA. In an embodiment, the target binding probe binds RNA. In some embodiments, the target binding probe binds a sugar. In some embodiments, the target binding probe binds a lipid. In an embodiment, the target binding probe binds a nucleic acid. In an embodiment, the target binding probe binds a particular covalent modification of a molecule. In an embodiment, the target binding probe comprises an antibody that binds a covalent modification of a protein. In an embodiment, the target binding probe comprises an antibody the binds a phosphorylated amino acid on a protein. In an embodiment, the target binding probe comprises an antibody the binds a methylated or an acetylated amino acid on a protein. In an embodiment, the target binding probe comprises an antibody that binds a carbohydrate, lipid, acetyl group, formyl group, acyl group, SUMO protein, Ubiquitin, Nedd or Prokaryotic ubiquitin-like protein on a protein of interest. In some embodiments, the proximity binding assay comprises contacting cellular material from single cells with target binding probes.

In some embodiments, the target binding probe comprises an antibody that binds to a target analyte. In certain embodiments, the target binding probe comprises an oligonucleotide that binds to a target analyte. In some embodiments, the target binding probe comprises an antibody conjugated with an oligonucleotide. In certain embodiments, the oligonucleotide comprises a sequence that binds preferentially to one or more bridging probes.

Oligonucleotides can be conjugated to antibodies by a number of methods known in the art (Kozlov et al., “Efficient strategies for the conjugation of oligonucleotides to antibodies enabling highly sensitive protein detection”; Biopolymers; 73(5); Apr. 5, 2004; pp. 621-630). Aldehydes can be introduced to antibodies by modification of primary amines or oxidation of carbohydrate residues. Aldehyde- or hydrazine-modified oligonucleotides are prepared either during phosphoramidite synthesis or by post-synthesis derivatization. Conjugation between the modified oligonucleotide and antibody result in the formation of a hydrazone bond that is stable over long periods of time under physiological conditions. Oligonucleotides can also be conjugated to antibodies by producing chemical handles through thiol/maleimide chemistry, azide/alkyne chemistry, tetrazine/cyclooctyne chemistry and other click chemistries. These chemical handles are prepared either during phosphoramidite synthesis or post-synthesis.

In some embodiments, between 2 and 50 different target binding probe pairs are used in a proximity binding assay, wherein each type of target binding probe pair detects a distinct target biomolecule. In certain embodiments, between 50 and 100, between 100 and 200, between 200 and 300, between 300 and 400, between 400 and 500, between 500 and 1,000, or between 1,000 and 10,000 distinct target binding probe pairs are used in a proximity binding assay.

In preferred embodiments, two antibodies or fragments thereof can be used to bind to a single target analyte of interest to improve accuracy of detection. Antibodies, though generated to bind unique antigens, often bind non-specifically to targets other than the target of interest. Such is frequently the case for polyclonal antibodies. In this example, one antibody may bind the target analyte, while also binding non-specifically to other antigens not of interest, thereby generating false positives if only one antibody is used. Including a second antibody, which itself may or may not bind non-specifically, but wherein only the target analyte of interest is bound by both antibodies, provides a method to accurately discriminate binding to the target analyte from non-specific binding. Thus, use of multiple antibodies in the proximity binding detection method can improve accurate identification and quantification of target analytes through reduction of false positives associated with background non-specific binding.

Aptamers and nucleic acid probes may also exhibit non-specific binding that in turn may result in false positives during analyte detection. As in the above example, use of two aptamers or two nucleic acid probes can improve accuracy of analyte identification and quantification by reducing the probability of false positives due to non-specific binding. In addition, the various target binding probe species can be mixed to improve accuracy, e.g. the use of an antibody in conjugation with the use of an aptamer or a nucleic acid probe, or a nucleic acid probe in conjugation with an aptamer, or an antibody, aptamer, or nucleic acid probe in conjugation with any other suitable target binding probe known to one skilled in the art.

In another embodiment, more than two target binding probes may be needed to accurately identify a target analyte. In this embodiment, repeated interrogation using proximity binding detection can performed wherein three or more total target binding probes are used. Following the example above, many cell types can only be identified when characterized by three or more surface features. Repeated interrogation can be performed using antibodies to additional surface features and the detection results combined to accurately identify specific cells.

Bridging Probes

Bridging probes, as discussed herein, primarily function to generate a detectable signal when a target binding probe pair is bound to the target analyte, as part of the proximity binding detection assay. Thus, in some embodiments, a bridging probe is a molecule or a complex having two binding sites to separately bind to each target binding probe when they are in proximity, and also having a detectable marker capable of generating a detectable signal. Sets of bridging probes can be provided for multiplexed detection of several target analytes over several cycles to generate multiple signal detection sequences for each target analyte bound to the surface of a substrate. In preferred embodiments, each set of bridging probes include bridging probes with the same binding moieties, but different detectable markers to facilitate generation of a heterogeneous signal sequence. This signal sequence includes redundant data to allow for recognition of a target analyte despite one or more incorrect signals.

In preferred embodiments, the bridging probe includes an oligonucleotide comprising two complementary regions, a first region complementary to a specificity determining oligonucleotide on a first probe of a target binding probe pair, and a second region complementary to a specificity determining oligonucleotide on a second probe of a target binding probe. In this embodiment, binding of the bridging probe to the pair of target binding probes occurs via nucleic acid hybridization of complementary sequences. Binding affinities between nucleotide pairs are well-known, such that conditions can be provided that facilitate removal of singly bound, but not doubly bound bridging probes. In some embodiments, the oligonucleotides comprise DNA, RNA, or PNA. Although complementary oligonucleotides are preferred, any binding moiety that specifically or preferentially binds to a target binding molecule under the conditions provided can be used in a bridging probe that binds to two target binding probes in proximity. This can include aptamers, antibodies, and other binding interactions where specific binding can occur, and the binding interaction can be reversed under selected conditions for cycled detection.

In some embodiments, the complementary region is 24 nucleotides in length. In some embodiments, the complementary region is 30, 40, 50, 60, 70, 80, 90 or 100 nucleotides in length. In some embodiments, the complementary region is from 24-30, from 24-40, from 24-50, from 24-60, from 24-70, from 24-80, from 24-90, or from 24-100 nucleotides in length. In some embodiments, the complementary region is 100 nucleotides in length or more.

In some embodiments, the detectable marker is directly or indirectly bound to the bridging probe oligonucleotide. In some embodiments, the detectable marker is hybridized to, conjugated to, or covalently linked to the bridging probe oligonucleotide. In some embodiments, the detectable marker is an optically detectable label, such as a fluorophore. In other embodiments, the detectable marker comprises an oligonucleotide sequence that has a homopolymeric base region (e.g., a poly-A tail). The bridging probe can be detected electrically, optically, or chemically via the detectable marker.

Detectable Marker

Each bridging probe includes a detectable marker. Following the removal of non-specifically or partially bound bridging probes, the detectable markers that remain bound to target analytes (via target binding probes) are detected during each cycle.

The target identification detectable marker can be any molecule capable of producing a signal for detecting a target biomolecule. Detectable markers include, but are not limited to, fluorophores, homopolymeric tails, or enzymes that catalyze a detectable signal. Detectable markers can be attached to bridging probes by means known to those skilled in the art. In some embodiments, a detectable marker comprises a fluorescent molecule, a chemiluminescent molecule, a chromophore, an enzyme, an enzyme substrate, an enzyme cofactor, an enzyme inhibitor, a dye, a metal ion, a metal sol, a ligand (e.g., biotin, avidin, streptavidin or haptens), radioactive isotope, and the like, and combinations thereof.

Optical detection methods can be used to quantify and identify a large number of analytes simultaneously in a sample. Optical detection methods used herein have previously been described in PCT Publication No. WO 2014/078855, “Digital Analysis of Molecular Analytes Using Single Molecule Detection,” incorporated by reference in its entirety.

In one embodiment, optical detection of fluorescently-tagged bridging probes can be achieved by frequency-modulated absorption and laser-induced fluorescence. Fluorescence can be more sensitive because it is intrinsically amplified as each fluorophore emits thousands to perhaps a million photons before it is photobleached. Fluorescence emission usually occurs in a four-step cycle: 1) electronic transition from the ground-electronic state to an excited-electronic state, the rate of which is a linear function of excitation power, b) internal relaxation in the excited-electronic state, c) radiative or non-radiative decay from the excited state to the ground state as determined by the excited state lifetime, and d) internal relaxation in the ground state. Single molecule fluorescence measurements are considered digital in nature because the measurement relies on a signal/no signal readout independent of the intensity of the signal.

Detectable markers can be attached chemically or covalently to any appropriate region of the target detection probe. In some embodiments, the detectable markers are fluorescent molecules. Fluorescent molecules can be fluorescent proteins or can be a reactive derivative of a fluorescent molecule known as a fluorophore. Fluorophores are fluorescent chemical compounds that emit light upon light excitation. In some embodiments, the fluorophore selectively binds to a specific region or functional group on the target molecule and can be attached chemically or biologically. Examples of fluorescent tags include, but are not limited to, green fluorescent protein (GFP), yellow fluorescent protein (YFP), red fluorescent protein (RFP), cyan fluorescent protein (CFP), fluorescein, fluorescein isothiocyanate (FITC), tetramethylrhodamine isothiocyanate (TRITC), cyanine (Cy3), phycoerythrin (R-PE) 5,6-carboxymethyl fluorescein, (5-carboxyfluorescein-N-hydroxysuccinimide ester), Texas red, nitrobenz-2-oxa-1,3-diazol-4-yl (NBD), coumarin, dansyl chloride, and rhodamine (5,6-tetramethyl rhodamine).

Optical detection requires an optical detection instrument or reader to detect the signal from the labeled probes. U.S. Pat. Nos. 8,428,454 and 8,175,452, which are incorporated by reference in their entireties, describe exemplary imaging systems that can be used and methods to improve the systems to achieve sub-pixel alignment tolerances. In some embodiments, methods of aptamer-based microarray technology can be used. See Optimization of Aptamer Microarray Technology for Multiple Protein Targets, Analytica Chimica Acta 564 (2006).

The high dynamic-range analyte quantification methods of the invention allow the measurement of over 10,000 analytes from a biological sample. The method can quantify analytes with concentrations from about 1 ag/mL to about 50 mg/mL and produce a dynamic range of more than 10¹⁰. The optical signals are digitized, and analytes are identified based on a code (ID code, or signal detection sequence) of digital signals for each analyte.

As described above, target analytes or complexes are bound to a solid substrate, and bridging probes are bound to the analytes using the proximity detection binding assay. Each of the bridging probes comprises a detectable marker and specifically binds to a target analyte. In some embodiments, the tags are fluorescent molecules that emit the same fluorescent color, and the signals for additional fluorophores are detected at each subsequent pass. During a pass, a set of bridging probes comprising detectable markers are contacted with the substrate allowing them to hybridize to the specificity determining oligonucleotides associated with their targets. An image of the substrate is captured, and the detectable signals are analyzed from the image obtained after each pass. The information about the presence and/or absence of detectable signals is recorded for each detected position (e.g., target analyte) on the substrate.

In some embodiments, the invention comprises methods that include steps for detecting optical signals emitted from the probes comprising detectable markers, counting the signals emitted during multiple passes and/or multiple cycles at various positions on the substrate, and analyzing the signals as digital information using a K-bit based calculation to identify each target analyte on the substrate. Error correction can be used to account for errors in the optically-detected signals, as described below.

In some embodiments, a substrate is bound with analytes comprising N target analytes. To detect N target analytes, M cycles of probe binding and signal detection are chosen. Each of the M cycles includes X sets of distinct bridging probes, such that each set of bridging probes specifically binds to one of the N target analytes. In certain embodiments, there are N sets of bridging probes for the N target analytes.

In each cycle, there is a predetermined order for introducing the sets of bridging probes for each pass. In some embodiments, the predetermined order for the sets of bridging probes is a randomized order. In other embodiments, the predetermined order for the sets of bridging probes is a non-randomized order. In one embodiment, the non-random order can be chosen by a computer processor. The predetermined order is represented in a key for each target analyte. A key is generated that includes the order of the sets of bridging probes, and the order of the bridging probes is digitized in a code to identify each of the target analytes.

In some embodiments, each set of ordered bridging probes is associated with a distinct detectable marker for detecting the target analyte, and the number of distinct tags is less than the number of N target analytes. In that case, each N target analyte is matched with a sequence of M tags for the M cycles. The ordered sequence of tags is associated with the target analyte as an identifying code.

After the detection process, the signals from each bridging probe pool are counted, and the presence or absence of a signal and the color of the signal can be recorded for each position on the substrate.

From the detectable signals, K bits of information are obtained in each of M cycles for the N distinct target analytes. The K bits of information are used to determine L total bits of information, such that K×M=L bits of information and L>log₂ (N). The L bits of information are used to determine the identity (and presence or characteristic) of N distinct target analytes. If only one cycle (M=1) is performed, then K×1=L. However, multiple cycles (M>1) can be performed to generate more total bits of information L per analyte. Each subsequent cycle provides additional optical signal information that is used to identify the target analyte.

In practice, errors in the signals occur, and this confounds the accuracy of the identification of target analytes. For instance, bridging probes may bind the wrong targets (e.g., false positives) or fail to bind the correct targets (e.g., false negatives). As described above, the proximity binding detection method aims to correct the occurrence of false positives by setting a higher specificity threshold. Additionally, methods are provided, as described below, to account for errors in optical and electrical signal detection. Thus, in preferred embodiments, sufficient cycles are performed such that L includes redundant bits (additional bits of information that can form part or all of the redundant data) for error correction (i.e., L>log₂ (N)).

In certain embodiments the detection markers are configured for electronic detection. In some embodiments, target analytes are tagged with oligonucleotide tail regions and the oligonucleotide tags are detected using ion-sensitive field-effect transistors (ISFET, or a pH sensor), which measures hydrogen ion concentrations in solution. Methods for electrical detection of probes is described in PCT Publication No. WO 2014/078855, “Digital Analysis of Molecular Analytes Using Single Molecule Detection,” incorporated by reference in its entirety. ISFETs are also described in further detail in U.S. Pat. No. 7,948,015, filed on Dec. 14, 2007, to Rothberg et al., and U.S. Publication No. 2010/0301398, filed on May 29, 2009, to Rothberg et al., which are each incorporated by reference in their entireties.

The electrical output signal detected from each cycle is digitized into bits of information, so that after all cycles have been performed to bind each tail region to its corresponding linker region, the total bits of obtained digital information can be used to identify and characterize the target biomolecule in question. The total number of bits is dependent on a number of identification bits for identification of the target biomolecule, plus a number of bits for error correction. The number of bits for error correction (i.e., redundant bits) can be selected based on the desired robustness and accuracy of the electrical output signal. Generally, the number of error correction bits will be 2 or 3 times the number of identification bits.

Cycled Detection and Error Correction

In optical and electrical detection methods described herein, errors can occur in binding and/or detection of signals. In bulk phase measurements, individual discrepancies in binding interactions are unlikely to significantly impact final measurements. However, when performing single molecule or single complex identification, as described herein, a single error can result in a misidentification, such as in a false negative or a false positive. In some cases, especially where target analyte populations or target analyte modifications represent a small, but important proportion of the total population, these errors can lead to undesirable results, such as misdiagnosis. Thus, improved accuracy of detection is an important aspect of single molecule detection and preferred embodiments of the invention described herein.

In some cases, the error rate can be as high as one in five (e.g., one out of five fluorescent signals is incorrect). This equates to one error in every five-cycle sequence. Actual error rates may not be as high as 20%, but error rates of a few percent are possible. In general, the error rate depends on many factors including the type of analytes in the sample and the type of probes used. In an electrical detection method, for example, a tail region may not properly bind to the corresponding probe region on an aptamer during a cycle. In an optical detection method, an antibody probe may not bind to its target or bind to the wrong target.

Thus, in preferred embodiments, the methods described herein included cycled repetition of detection with ordered probe sets to generate a uniquely identifiable code with redundant data that is associated with the target analyte or a modification thereof. Cycle repetition involves repeated interrogation of the target analyte to reduce that rate of false positives and false negatives that may occur during the proximity binding detection method. Methods for cycle repetition are described in WO 2014/078855, “Digital Analysis of Molecular Analytes Using Single Molecule Detection,” incorporated by reference in its entirety.

The target detection probes and/or bridging probes used to detect the target analytes are introduced to the substrate in an ordered manner in each cycle. After the detection process, the signals from each probe pool are counted, and the presence or absence of a signal and the color of the signal can be recorded for each position on the substrate. The signals detected for each target analyte can be digitized into bits of information. The order of the signals provides a code for identifying each analyte/target biomolecule and/or cell of origin, which can be encoded in bits of information. The code can be compared to a generated key that encodes information about the order of the probes for each target analyte.

In preferred embodiments, the bridging probe binding and detection cycle is repeated using new bridging probes. In this example, the previous bridging probes are removed without removing the target binding probes. Removal is carried out using methods known to those skilled in the art, including, but not limited to, use of heat, denaturation agents, salts, detergents etc. Following removal, new bridging probes are added. The new bridging probes are again engineered to hybridize to the specificity determining oligonucleotides associated with each target binding probe. The new bridging probes may be conjugated to a new detectable marker or conjugated to the same detectable marker. In one embodiment, a new bridging probe specific for one target analyte is conjugated to a new detectable marker, while another bridging probe specific for a second target analyte is conjugated to the same detectable marker. Following addition of the new bridging probes, the sample is washed and detected, as described above.

Following detection of the detectable marker, in some embodiments, the cycle for detection is repeated by stripping both the bridging probes and the target binding probes. Removal is carried out using methods known to those skilled in the art, including, but not limited to, use of heat, denaturation agents, salts, detergents etc. Following addition of new target binding probes, bridging probes specific for the specificity determining oligonucleotides conjugated to the new target binding probes are added, washed, and detected, as described above. Alternatively, if the new target binding probes are distinct from those in previous cycles, i.e., they bind to different epitopes of the target analyte or complex, the new target binding probes can be added without removal of the previous target binding probes (while the bridging probes are still removed to avoid interference with the next cycle of detection).

In some embodiments, the conditions used to remove target binding probes or bridging probes take into consideration maintaining complexes or the native conformation of molecules. In some embodiments, the wash conditions take into consideration avoiding removal of the specifically bound target binding probes. In some embodiments, the wash conditions take into consideration if two types of target binding probes are used, for example, an antibody used in conjunction with a nucleic acid probe.

When performing cycles of detection, the total bits of information obtained (L) can be defined by the number of bits per cycle (K) multiplied by the number of cycles (M) [L=K×M]. The total number of bits (L) required to identify the total number of analytes N without redundant data is defined by L=log₂N. Thus, L total bits of information must be acquired to generate information for N total analytes. The L total bits of information is dependent upon the number of bits per cycle (K) and the total number of cycles (M).

Herein, we describe a cycled method of detection that generates a detection signal sequence that includes redundant data for error correction during detection. Thus, the total bits of information collected, including redundant data, must be greater than log 2N. In preferred embodiments to reduce detection error, the number of cycles performed and the number of bits per cycle collected are such that K×M>log₂N (i.e., L>log₂N). This relationship governs the physical steps of the method required to iterate the number of cycles performed and the number of bits of information collected by each set of bridging probes for each cycle. Thus, to incorporate error correction, additional cycles are generated to account for errors in the detected signals and to obtain additional data, i.e., redundant data, which can comprise additional bits of information, (i.e., redundant bits).

The additional data, which can include the additional bits of information, are used to correct errors (e.g., false positives and/or false negatives) and/or validate detection data using an error-correcting code. In one embodiment, the error-correcting code is a forward error correction code (FEC). In one embodiment, the error-correcting code is a Reed-Solomon code, which is a non-binary cyclic code used to detect and correct errors in a system. In other embodiments, various other error-correcting codes can be used. Other error-correcting codes include, for example, block codes, convolution codes, Golay codes, Hamming codes, BCH codes, AN codes, Reed-Muller codes, Goppa codes, Hadamard codes, Walsh codes, Hagelbarger codes, polar codes, repetition codes, repeat-accumulate codes, erasure codes, online codes, group codes, expander codes, constant-weight codes, tornado codes, low-density parity check codes, maximum distance codes, burst error codes, luby transform codes, fountain codes, and raptor codes. See Error Control Coding, 2^{nd} Ed., S. Lin and DJ Costello, Prentice Hall, New York, 2004. Methods for error correction are described in PCT Publication No. WO 2014/078855, “Digital Analysis of Molecular Analytes Using Single Molecule Detection,” incorporated by reference in its entirety.

In certain embodiments, error correction can reduce the false-positive detection rate to less than 1 in 10⁴, less than 1 in 10⁵, less than 1 in 10⁷, less than 1 in 10⁸ or less than 1 in 10⁹. In certain embodiments, error correction can reduce the false-negative detection rate to less than 1 in 10⁴, less than 1 in 10⁵, less than 1 in 10⁷, less than 1 in 10⁸ or less than 1 in 10⁹.

In certain aspects, the target analyte proximity binding assay comprises determining L total bits of information such that L is sufficient to reduce a false positive error rate of detection to less than 1 in 10⁶. In certain aspects, the false-positive detection rate is less than less than 1 in 10⁴, 1 in 10⁵, less than 1 in 10⁷, less than 1 in 10⁸ or less than 1 in 10⁹. In an aspect, L is a function of the misidentification rate for a target biomolecule at each cycle. In an aspect, the misidentification rate comprises the non-binding rate and the false binding rate of the probe to the target biomolecule. In certain aspects, L comprises bits of information that are ordered in a predetermined order. In certain aspects, the predetermined order is a random order. In certain aspects, L comprises bits of information comprising a key for decoding an order of the plurality of ordered target detection probe set and/or cell identifier probe set. In certain aspects, at least K bits of information comprise information about the absence of a signal for one of the N distinct target biomolecules.

In certain aspects, successful detection is achieved using bridging probes and/or target detection probes have a cross-reactivity with non-target biomolecule of greater than 2%, 5%, 10%, 15%, 20%, or 25%. In certain aspects, successful detection is achieved where at least one of the target analytes does not bind to a corresponding cell identifier probe and/or target detection probe for at least 10%, at least 20%, at least 30%, or at least 40% of cycles.

It is also contemplated that the proximity binding detection method can be highly multiplexed, i.e. that multiple target analytes can be simultaneously interrogated on a substrate through use of multiple distinct bridging probes, each distinct bridging probe specific for a distinct target analyte.

In another embodiment, multiple rounds of interrogation can be performed to determine total target analyte, whether a target analyte is modified, and/or whether a target analyte is unmodified. In another embodiment, multiple rounds of interrogation can be performed to determine the ratio between modified, unmodified and total target analytes. For example, one or more rounds of proximity binding detection can be used to accurately identify and quantify modified target analytes. Additional rounds can be performed to accurately identify and quantify total target analytes and the ratio of modified to total target analyte quantified. In another embodiment, one or more rounds of proximity binding detection can be used to accurately identify and quantify modified target analytes. Additional rounds can be performed to accurately identify and quantify unmodified target analytes and the ratio of modified to unmodified target analyte quantified. In another embodiment, one or more rounds of proximity binding detection can be used to accurately identify and quantify unmodified target analytes. Additional rounds can be performed to accurately identify and quantify total target analytes and the ratio of unmodified to total target analyte quantified.

In another embodiment, the proximity binding detection method can be used in conjunction with other detection methods to accurately identify and quantify target analytes. For example, repeated interrogation can be performed wherein one or more rounds of interrogation uses the proximity binding detection method, while another round(s) uses a standard target binding probe covalently linked to a detectable marker, and the detection results combined to accurately identify and quantify target analytes.

### OTHER EMBODIMENTS

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3rd Ed. (Plenum Press) Vols A and B (1992).

1. A method for identifying a presence or absence of one or more distinct target analytes in a sample, comprising:
i) distributing a sample suspected of comprising N distinct target analytes on a substrate such that the target analytes, if present, bind to the substrate at spatially separate regions;
ii) contacting said sample with N distinct binding probe pairs, wherein each of said N distinct binding probe pairs comprises a first target binding probe and a second target binding probe, wherein said first target binding probe comprises a first specificity determining oligonucleotide, and wherein said second target binding probe comprises a second specificity determining oligonucleotide, wherein said first and second target binding probes are configured to selectively bind as a pair to one of said N distinct target analytes;
iii) performing M cycles of analyte detection, wherein M is greater than 1, thereby generating a signal detection sequence from one or more of said spatially separate regions, wherein said signal detection sequence comprises redundant data for error correction, each cycle comprising:
contacting said sample with an ordered detection probe reagent set comprising X distinct bridging probes each comprising a detectable marker, a first bridging probe oligonucleotide complementary to said first specificity determining oligonucleotide of at least one of said N distinct binding probe pairs, and a second bridging probe oligonucleotide complementary to said second specificity determining oligonucleotide of said at least one of said N distinct binding probe pairs;
washing said substrate to remove said bridging probes that are not bound to one of said N distinct binding probe pairs;
detecting a presence or absence of a signal from said detectable marker at the spatially separate regions; and
if another cycle is to be performed, exposing said substrate to conditions capable of removing said bridging probe from said target analytes; and
iv) analyzing the signal detection sequence to identify the presence or absence of the one or more distinct target analytes in said sample.

2. (canceled)

3. (canceled)

4. (canceled)

5. The method of claim 1, wherein performing said M cycles of analyte detection generates at least K bits of information per cycle for said N distinct target analytes, wherein said at least K bits of information are used to determine L total bits of information, wherein K×M=L bits of information and L>log 2 (N), wherein said L bits of information are used to determine the presence or absence of said N distinct target analytes, wherein K=log₂(X), and wherein X<N or X=N.

6. (canceled)

7. (canceled)

8. (canceled)

9. (canceled)

10. The method of claim 1, wherein said first and second bridging probe and specificity determining oligonucleotides comprise DNA, RNA, PNA, or LNA.

11. (canceled)

12. (canceled)

13. (canceled)

14. (canceled)

15. (canceled)

16. The method of claim 1, wherein said sample comprises cell extracts, body fluids, biological specimen, biological culture, biological lysate, immunoprecipitated proteins, animal extracts, plant extracts, microbial organism extracts, toxins, allergens, hormones, steroids, cytokines, methylated proteins, phosphorylated proteins, acetylated proteins, immuno-precipitated protein complexes, or any combination thereof.

17. (canceled)

18. (canceled)

19. (canceled)

20. (canceled)

21. (canceled)

22. (canceled)

23. The method of claim 1, wherein said one or more distinct target analytes comprise a single protein polypeptide, protein complex polypeptide, polynucleotide, toxins, allergens, hormones, steroids, cytokines, or any combination thereof.

24. (canceled)

25. (canceled)

26. (canceled)

27. The method of claim 1, wherein at least one of said N distinct target analytes is a single molecule, protein-protein complex cross-linked with reversible linkers, protein-protein complex cross-linked with irreversible linkers, protein-nucleic acid complex cross-linked with reversible linkers, protein-nucleic acid complex cross-linked with irreversible linkers, or any combination thereof.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. The method of claim 1, wherein removing said bridging probe comprises separating the first and second specificity determining oligonucleotides from their respective first and second bridging probe oligonucleotides, wherein said separating comprises denaturing the sample by heat, denaturation agents, salts, detergents or any combination thereof.

46. (canceled)

47. (canceled)

48. (canceled)

49. (canceled)

50. (canceled)

51. (canceled)

52. A method for identifying a presence or absence of one or more distinct target analytes in a sample, comprising:
i) contacting a sample suspected of comprising N distinct target analytes with N distinct binding probe pairs, wherein each of said N distinct binding probe pairs comprises a first target binding probe and a second target binding probe, wherein said first target binding probe comprises a first specificity determining oligonucleotide, and wherein said second target binding probe comprises a second specificity determining oligonucleotide, wherein said first and second target binding probes are configured to selectively bind as a pair to one of said N distinct target analytes;
ii) contacting said sample with a detection probe reagent set comprising N distinct bridging probes each comprising a functional substrate binding group, a first bridging probe oligonucleotide complementary to said first specificity determining oligonucleotide of at least one of said N distinct binding probe pairs, and a second bridging probe oligonucleotide complementary to said second specificity determining oligonucleotide of said at least one of said N distinct binding probe pairs;
iii) removing unbound bridging probes from said sample;
iv) distributing said sample on a substrate such that target-analyte bound bridging probes bind to the surface of said substrate via said functional substrate binding group at spatially separate regions of said substrate;
v) performing M cycles of analyte detection, wherein M is greater than 1, thereby generating a signal detection sequence from one or more of said spatially separate regions, wherein said signal detection sequence comprises redundant data for error correction, each cycle comprising:
contacting said sample with an ordered probe reagent set comprising X distinct probes each comprising a detectable marker and a sequence complementary to one of said N distinct bridging probes;
washing said substrate to remove unbound probes;
detecting a presence or absence of a signal from said detectable marker at the spatially separate regions; and
if another cycle is to be performed, exposing said substrate to conditions capable of removing said bridging probe from said target analytes; and
vi) analyzing the signal detection sequence to identify the presence or absence of the one or more distinct target analytes in said sample.

53. The method of claim 52, wherein performing said M cycles of analyte detection generates at least K bits of information per cycle for said N distinct target analytes, wherein said at least K bits of information are used to determine L total bits of information, wherein K×M=L bits of information and L>log 2 (N), wherein said L bits of information are used to determine the presence or absence of said N distinct target analytes, wherein K=log₂(X), and wherein X<N or X=N.

54. The method claim 52, wherein said first and second bridging probe and specificity determining oligonucleotides comprise DNA, RNA, PNA, or LNA.

55. The method claim 52, wherein said sample comprises cell extracts, body fluids, biological specimen, biological culture, biological lysate, immunoprecipitated proteins, animal extracts, plant extracts, microbial organism extracts, toxins, allergens, hormones, steroids, cytokines, methylated proteins, phosphorylated proteins, acetylated proteins, immuno-precipitated protein complexes, or any combination thereof.

56. The method of claim 52, wherein said one or more distinct target analytes comprise a single protein polypeptide, protein complex polypeptide, polynucleotide, toxins, allergens, hormones, steroids, cytokines, or any combination thereof.

57. The method of claim 52, wherein at least one of said N distinct target analytes is a single molecule, protein-protein complex cross-linked with reversible linkers, protein-protein complex cross-linked with irreversible linkers, protein-nucleic acid complex cross-linked with reversible linkers, protein-nucleic acid complex cross-linked with irreversible linkers, or any combination thereof.

58. The method of claim 52, wherein removing said bridging probe comprises separating the first and second specificity determining oligonucleotides from their respective first and second bridging probe oligonucleotides, wherein said separating comprises denaturing the sample by heat, denaturation agents, salts, detergents or any combination thereof.

59. A composition for detecting an analyte, comprising:
a pair of target binding probes, wherein the target binding probes are configured to specifically bind to a target analyte; and
a bridging probe, wherein the bridging probe comprises a binding site to bind to said target binding probe and a detectable marker capable of generating a detectable signal.

60. The composition of claim 59, wherein said pair of target binding probes comprises antibodies.

61. The composition of claim 59, wherein said pair of target binding probes comprises aptamers

62. The composition of claim 59, wherein said pair of targeted binding probes comprises nucleic acid probes.

63. The composition of claim 59, wherein said pair of target binding probes are not the same.

64. The composition of claim 60, wherein said antibodies bind to a carbohydrate, lipid, acetyl group, formyl group, acyl group, SUMO protein, Ubiquitin, Nedd or Prokaryotic ubiquitin-like protein on a protein of interest.

65. The composition of claim 59, wherein said target analyte comprises DNA, RNA, sugar, lipid, nucleic acid, covalent modification of a protein, phosphorylated amino acid on a protein, methylated or an acetylated amino acid on a protein

66. The composition of claim 59, wherein said bridging probes comprises two binding sites.

67. The composition of claim 59, wherein said bridging probe binding comprises a set of bridging probes.
